(19) United States
(12) Patent Application Publication
(10) Pub. No.: US 2018/0174692 A1
(43) Pub. Date: Jun. 21, 2018

(54) System and Method of Managing and Coordinating a Medical Procedure

(71) Applicant: Higgs Boson, LLC, Durham, NC (US)

(72) Inventors: Shivanand Lad, Durham, NC (US); Ziad Gellad, Durham, NC (US); Joseph Knight, Durham, NC (US); Rajeev Dharmapurikar, Durham, NC (US)

(21) Appl. No.: 15/873,191

(22) Filed: Jan. 17, 2018

Related U.S. Application Data
(60) Provisional application No. 62/423,681, filed on Nov. 17, 2016.

(51) Int. Cl. G16H 80/00; G16H 10/60

(52) U.S. Cl. CPC G16H 80/00; G16H 40/20; G16H 10/60

(57) ABSTRACT

A system and method of managing and coordinating a medical procedure simplifies a desired medical procedure from beginning to end. The method includes at least one practitioner account and at least one patient account, both managed by at least one remote server. The method allows the practitioner account and the patient account to communicate with each other through a practitioner personal computing (PC) device and a patient PC device, respectively. The method begins with the execution of an exploratory process in order to identify a desired medical procedure. A distal preparatory process is then executed to gather and manage pre-procedure logistic data. A proximal preparatory process is then executed in order to gather and manage pre-procedure checklist data for the desired medical procedure. The method is completed with execution of a recovery process in order to gather and manage post-procedure logistic data for the desired medical procedure.

The current application claims priority to U.S. provisional application serial No. 62/423,681 filed on Nov. 17, 2016.

### FIELD OF THE INVENTION

The present invention generally relates to managing and coordinating a medical procedure. More specifically, the present invention simplifies the preparation and healing processes of demanding medical procedures using input provided from a computing device.

### BACKGROUND OF THE INVENTION

According to the Center for Diseases Control and Prevention (CDC) more than fifty million surgical procedures are conducted in the United States on an annual basis.

While there is no exact data for medical procedures on a global scale, it is logical to assume the number is extremely high due to the sheer size of the population. Most patients experience anxiety before medical procedures, especially complex procedures that require continuous involvement. More than half of surgical errors occur outside the operation room. Traditionally, patient checklists are being used in hospitals to monitor the procedural steps before and after a medical procedure. Due to the high rate of miscommunication and the ease with which these checklists can be misplaced, improving Patient Activation Measure (PAM) would result in a more effective encounter between the patient and their medical provider. PAM is commonly defined as the ability of a patient to monitor their own progress through the rehabilitation process. The well-being of a patient does not only depend on the quality of care provided by the medical staff, but also their own ability to keep track of their own condition. Often times a patient may not make an appointment due to a variety of issues, such as transportation availability, pain, or other factors. The availability of a physician is a very scarce resource, therefore optimizing the process which needs to be conducted before an in-person meeting with a patient, would result in a better quality of care. Disorganized and misinformed patients can affect the quality of care they receive. For example, a patient might stop the post-procedure check-ups when they start feeling better, thus allowing for critical problems to develop when preventive measures could have been enforced at an earlier stage.

To overcome the issues shown above, the present invention aims to solve the common challenges faced by surgery patients. Multiple healthcare systems have been developed in an effort to address the common issues faced by care providers, patients and their families. Furthermore, this problem is amplified in developing countries. In places where basic medical supplies are scarce, the lack of support systems and facilities for physicians hinders the patient recovery process. Utilizing the available technologies, the present invention aims to maximize the efficiency of the limited time duration of patient visits, reduce the chances for error, and direct the patient towards following a recovery plan through their experience to improve healthcare engagement and outcomes. Computing devices, such as laptops, desktop computer, smartphones, and tablets, can provide patients, physicians, pharmaceutical companies, and medical device manufacturers input from the users in a variety of ways.

The present invention is a mobile application healthcare based process. It combines a mobile application and server based communication with a patient's and healthcare provider's mobile devices, allowing for effective communication and data transfer. The present invention may include, for example, formulating and rendering post-surgery care management plans, collecting and reporting information from patients and providers, data collection from patient/provider via the mobile device, and other functions pertaining to the visit of the patient, including the patient use of post-surgery data and follow up care, educational materials, and the ability to utilize the system. Furthermore, the present invention provides a method of organization which includes reminders, checklist tasks, and procedure specific information integrated into one platform.

### BRIEF DESCRIPTION OF THE DRAWINGS

FIG. 1 is a diagram illustrating electronic interactions in the present invention.

FIG. 2 is a flowchart illustrating the overall process for the present invention.

FIG. 3 is a flowchart illustrating the process for updating the practitioner account from an unverified status to a verified status.

FIG. 4 is a flowchart illustrating the process for granting access to the patient account to view the practitioner account profile.

FIG. 5 is a flowchart illustrating the process for initiating step (C) with the remote server, if the patient accepts an engagement invitation.

FIG. 6 is a flowchart illustrating the process for restricting access to the practitioner account from viewing a case study.

FIG. 7 is a flowchart illustrating the process for prompting the patient account to accept or deny the agency recommendation and repeating steps (C) through (F) with the other account as the practitioner account.

FIG. 8 is a flowchart illustrating the process for prompting the patient account to send a task confirmation to the practitioner account and displaying the task confirmation through the practitioner portable computing (PC) device for the distal preparatory process.

FIG. 9 is a flowchart illustrating the process for prompting the patient account to send a task confirmation to the practitioner account and displaying the task confirmation through the practitioner portable computing (PC) device for the proximal preparatory process.

FIG. 10 is a flowchart illustrating the process for prompting the patient account to send a task confirmation to the practitioner account and displaying the task confirmation through the practitioner portable computing (PC) device for the recovery preparatory process.

FIG. 11 is a flowchart illustrating the process for relaying a message from the practitioner PC device, through the remote server, and to the patient account.

FIG. 12 is a flowchart illustrating the process for relaying a message from the patient PC device, through the remote server, and to the practitioner account.

FIG. 13 is a flowchart illustrating the process for displaying a desired piece of information for the exploratory process.

FIG. 14 is a flowchart illustrating the process for displaying a desired piece of information for the distal preparatory process.

FIG. 15 is a flowchart illustrating the process for displaying a desired piece of information for the proximal preparatory process.

FIG. 16 is a flowchart illustrating the process for displaying a desired piece of information for the recovery process.

FIG. 17 is a flowchart illustrating the process for applying an organization filter to the practitioner task list.

FIG. 18 is a flowchart illustrating the process for appending a new entry from the patient PC device into a recovery log that is managed by the remote server.

FIG. 19 is a flowchart illustrating the process for sending a healthcare-accepted notification to the patient PC device and the practitioner PC device.

FIG. 20 is a flowchart illustrating the process for sending a financial payment plan to the patient PC device.

### DETAIL DESCRIPTIONS OF THE INVENTION

All illustrations of the drawings are for the purpose of describing selected versions of the present invention and are not intended to limit the scope of the present invention.

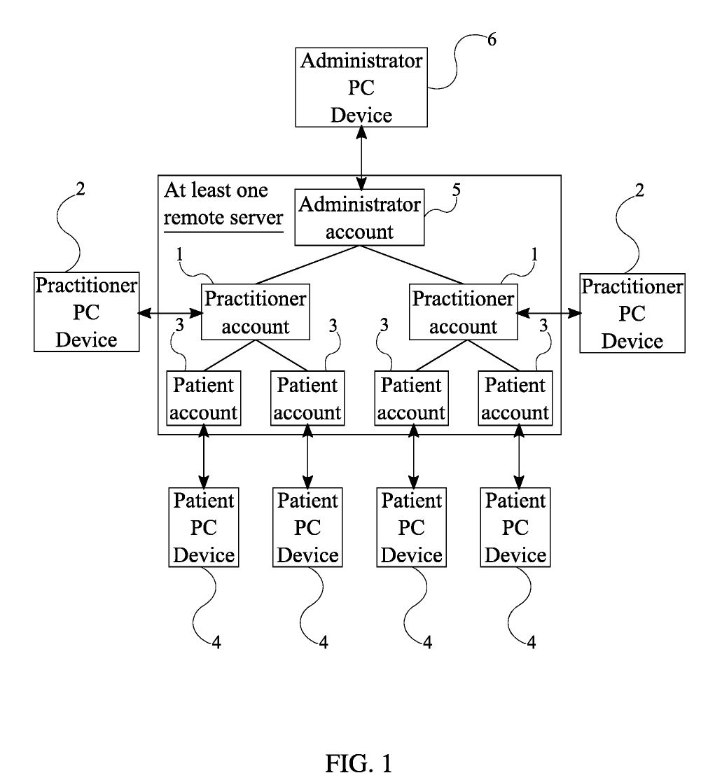

The present invention is a system and method managing and coordinating a medical procedure. The present invention is used to simplify the preparation and recovery processes of invasive medical procedures. The present invention may also aid in the preparation and recovery processes of non-invasive procedures. The present invention aids in the proper and thorough execution of tasks, as well as the accurate recording of information regarding the physical and mental status of a patient. Thus, the physical system used to implement the method of the present invention includes at least one remote server, a practitioner personal computing (PC) device 2, and a patient PC device 4, illustrated in FIG. 1. At least one practitioner account 1 is managed by the at least one remote server, wherein the practitioner account 1 is associated to the practitioner PC device 2 (Step A). Any data associated with the at least one practitioner account 1 is stored on the at least one remote server, and any inputs from the practitioner PC device 2 is executed via the at least one remote server. Similarly, at least one patient account 3 is managed by the remote server, wherein the patient account 3 is associated to a patient PC device 4 (Step B). Any data associated with the at least one patient account 3 is stored on the at least one remote server, and any inputs from the patient PC device 4 is executed via the at least one remote server. The patient PC device 4 and the practitioner PC device 2 communicate with each other via the at least one remote server.

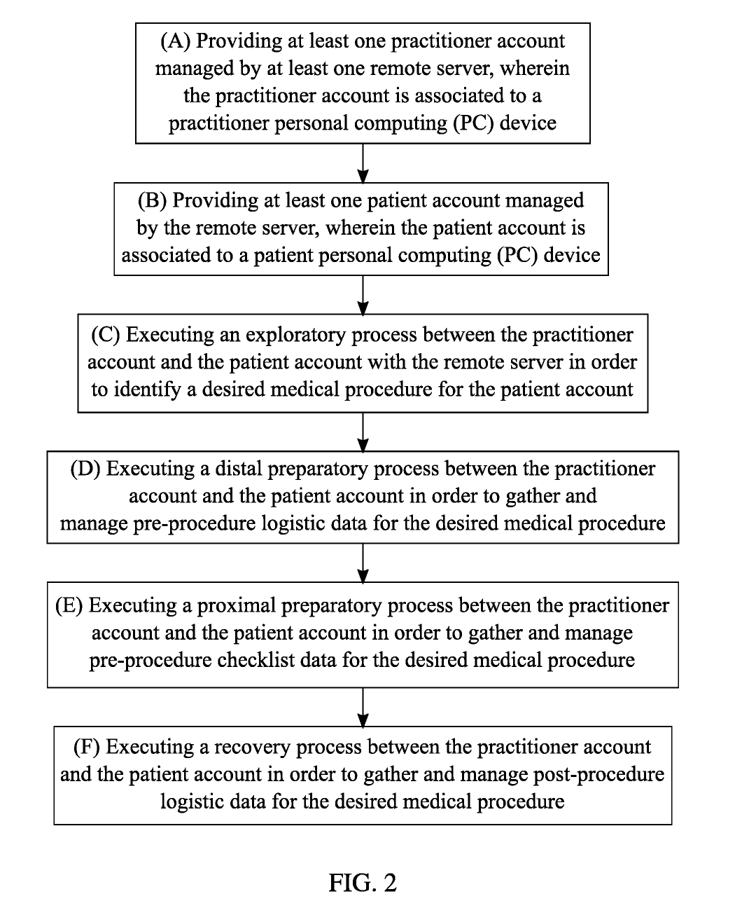

The overall process for the present invention includes the following steps that are implemented with the at least one remote server, the practitioner personal computing (PC) device 2, and the patient PC device 4. As shown in FIG. 2, the overall process begins with the execution of an exploratory process between the practitioner account 1 and the patient account 3 with the remote server in order to identify a desired medical procedure for the patient account 3 (Step C). The exploratory process defines the data associated with the patient account 3 for the following processes. A distal preparatory process is executed between the practitioner account 1 and the patient account 3 in order to gather and manage pre-procedure logistic data for the desired medical procedure (Step D). The distal preparatory process prepares the patient for the desired medical procedure by ensuring the patient account 3 completes any requests as dictated by the practitioner account 1. Upon the day of the desired medical procedure, a proximal preparatory process is executed between the practitioner account 1 and the patient account 3 in order to gather and manage pre-procedure checklist data for the desired medical procedure (Step E). A recovery process is executed between the practitioner account 1 and the patient account 3 in order to gather and manage post-procedure logistic data for the desired medical procedure (Step F). The recovery process guides the patient account 3 to accurately record the recovery of the user of the patient account 3 and provides the user of the patient account 3 with motivation in order to facilitate the recovery process.

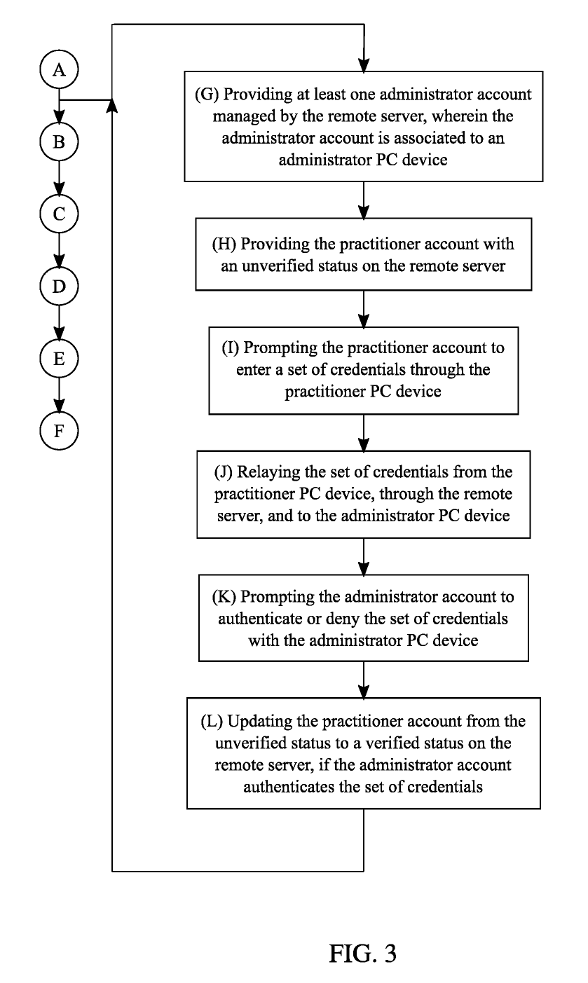

In order to verify and register a practitioner account 1, the physical system used to implement the method of the present invention further includes an administrator PC device 6, as seen in FIG. 3. At least one administrator account 5 is managed by the remote server, wherein the administrator account 5 is associated to the administrator PC device 6. The administrator account 5 requests specific inputs from the practitioner account 1 and verifies the inputs in order to confirm the identity of the practitioner account 1. The administrator account 5 limits unauthorized practitioners from falsely identifying as a licensed practitioner. Initially, the practitioner account 1 is provided with an unverified status on the remote server in order to differentiate between practitioner accounts 1. The practitioner account 1 is prompted to enter a set of credentials through the practitioner PC device 2 so that the administrator account 5 receives the corresponding set of credentials for the specific practitioner account 1. The set of credentials may include, but is not limited to, a license number, proof of a degree from an accredited graduate school, proof of an accredited business, and so on. The set of credentials from the practitioner PC device 2 is relayed through the remote server and to the administrator PC device 6 for verification by the administrator account 5. The administrator account 5 is prompted to authenticate or deny the set of credentials with the administrator PC device 6. The authentication or denial of the set of credentials prevents an unlicensed practitioner from identifying as a licensed practitioner with the present invention. If the administrator account 5 authenticates the set of credentials, the practitioner account 1 is updated from the unverified status to a verified status on the remote server. The verified status allows the user to identify as a practitioner and grants the user access to all the functions as a user of a practitioner account 1.

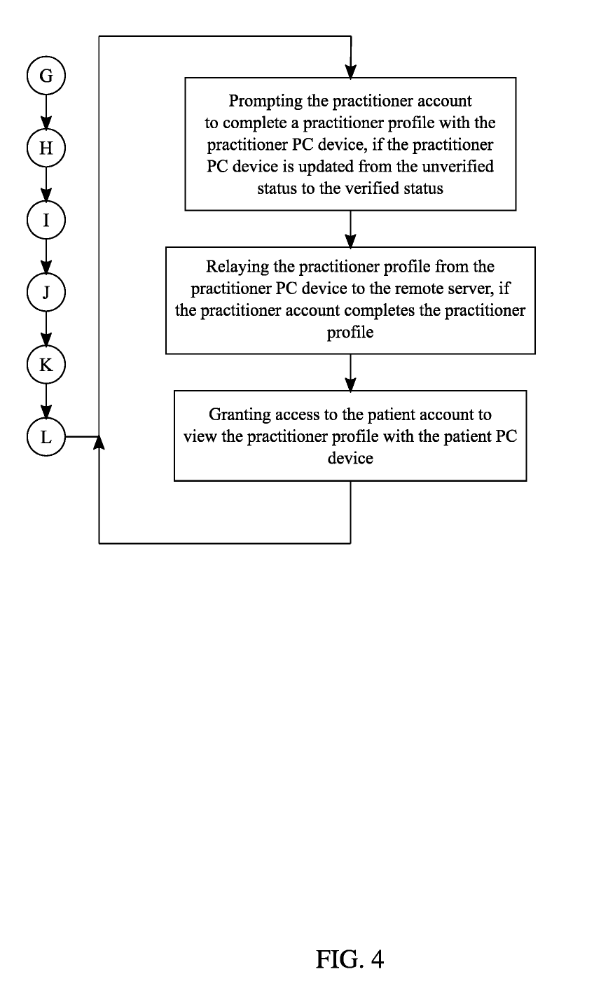

The at least one patient account 3 is able to view information regarding the practitioner account 1 via the patient PC device 4, also seen in FIG. 4, as the practitioner account 1 is prompted to complete a practitioner profile with the practitioner PC device 2, only if the practitioner PC device 2 is updated from the unverified status to the verified status. The practitioner profile requests inputs such as name of user, name of practice, email address, office hours, services provided, price of each service, and so on. The practitioner profile serves as a reference for the patient account 3 regarding general information of the practitioner account 1. If the practitioner account 1 completes the practitioner profile, the practitioner profile is then relayed from the practitioner PC device 2 to the remote server, thereby corresponding the information inputted through the practitioner PC device 2 to the practitioner profile. The patient account 3 is granted access to view the practitioner profile with the PC device, which the patient account 3 may review as needed.

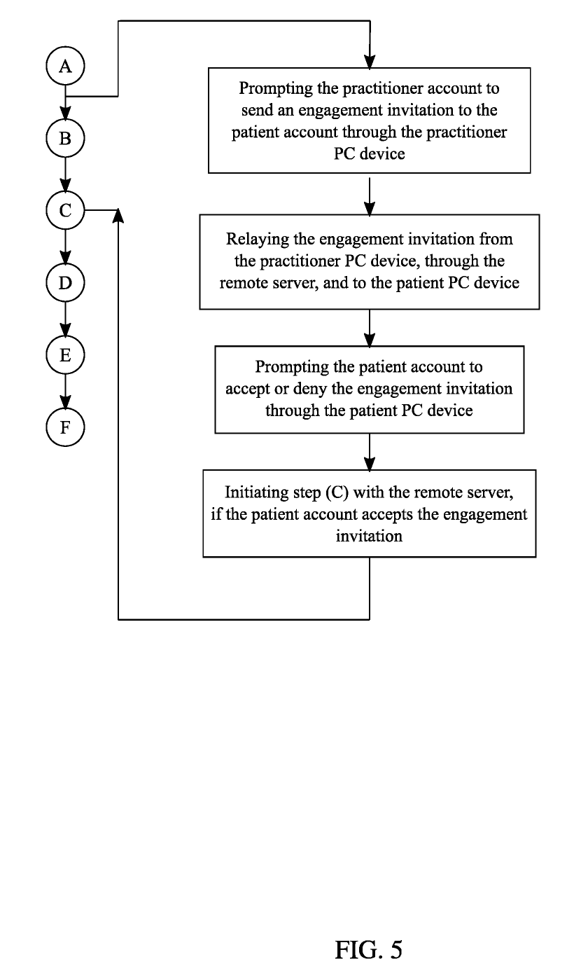

In order for at least one of the patient account 3 to be granted access to view the practitioner profile of at least one practitioner account 1, the practitioner account 1 is prompted to send an engagement invitation to the patient account 3 through the practitioner PC device 2, as seen in FIG. 5. The engagement invitation alerts the patient account 3 that the corresponding practitioner account 1 is attempting to establish a connection through the present invention. The engagement invitation from the practitioner PC device 2 is relayed through the remote server and to the patient PC device 4 so that the practitioner account 1 connects only with corresponding patient accounts 3 and more specifically, patient accounts 3 with a desired medical procedure that is invasive. The patient account 3 is prompted to accept or deny the engagement invitation through the patient PC device 4. If the patient account 3 accepts the engagement invitation, Step C is initiated and the patient account 3 is able to view the practitioner profile via the patient PC device 4 and to communicate with the practitioner account 1.

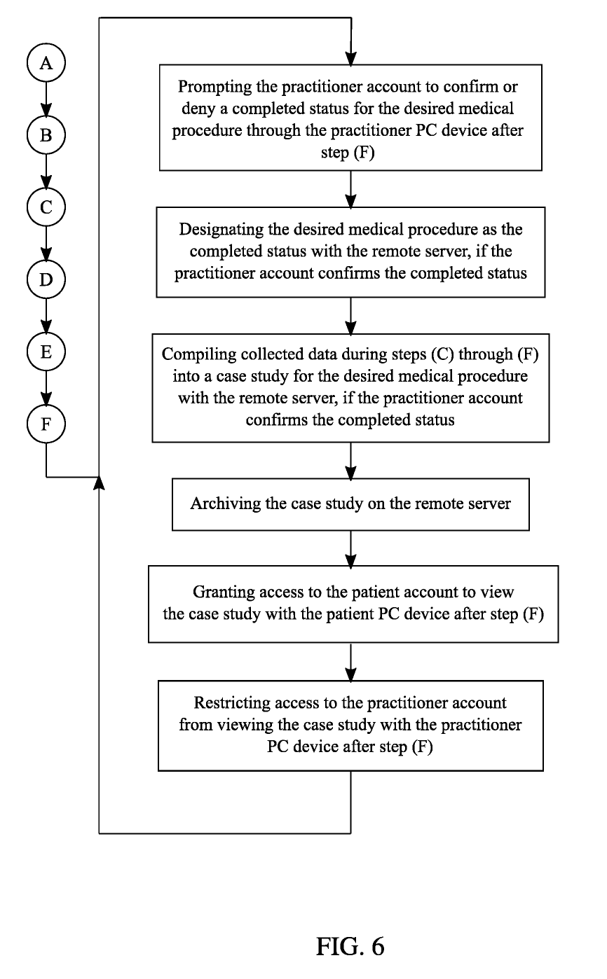

Upon the completion of the desired medical procedure, which includes the completion of the exploratory process, the distal preparatory process, the proximal preparatory process, and the recovery process, the practitioner account 1 is prompted to confirm or deny a completed status for the desired medical procedure through the practitioner PC device 2 after Step F. The desired medical procedure is designated as the completed status with the remote server, if the practitioner account 1 confirms the completed status, thereby preventing both the practitioner account 1 and the patient account 3 from inputting or altering any data corresponding to the desired medical procedure. The completed status also disconnects the established connection between the practitioner account 1 and the patient account 3 with respect to the desired medical procedure. As seen in FIG. 6, if the practitioner account 1 confirms the completed status, the patient account 3 is able to view a desired medical procedure with a completed status as the collected data during Steps C through F is compiled into a case study for the desired medical procedure with the remote server. More specifically, the case study is archived on the remote server and the patient account 3 is granted access to view the case study with the patient PC device 4 after Step F. The practitioner account 1 is restricted access from viewing the case study with the practitioner PC device 2 after Step F as the present invention does not serve as electronic medical records for the corresponding patient account 3.

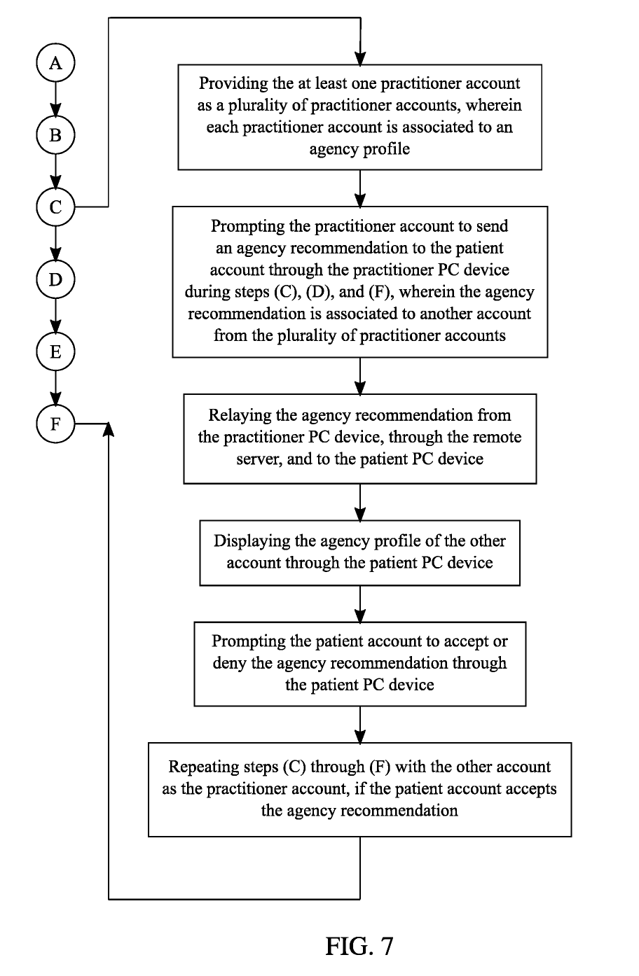

As seen in FIG. 7, in the event the patient account 3 needs to establish a connection with another practitioner due to certain medical conditions or medical needs, the at least one practitioner account 1 is provided as a plurality of practitioner accounts 1, wherein each practitioner account 1 is associated to an agency profile. The agency profile may include, but is not limited to, various practitioner accounts 1, a physical therapist account, a social worker account, a caretaker account, a dietitian account, a mental health therapist account, and so on. The practitioner account 1 provides a patient account 3 with options as the practitioner account 1 is prompted to send an agency recommendation to the patient account 3 through the practitioner PC device 2 during Steps C, D, and F, wherein the agency recommendation is associated to an other account from the plurality of practitioner accounts 1. The agency recommendation is relayed from the practitioner PC device 2, through the remote server, and to the patient PC device 4 so that the patient account 3 is able to view the options as defined by the practitioner account 1 through the patient PC device 4. The agency profile of the other account is displayed through the patient PC device 4 allowing the patient profile to make an educated choice regarding which agency account best suits the medical needs of the patient account 3. The patient account 3 is then prompted to accept or deny the agency recommendation through the patient PC device 4. If the patient account 3 accepts the agency recommendation, Steps C through F are repeated with the other account as the practitioner account 1 thereby establishing a connection with the agency recommendation with the present invention.

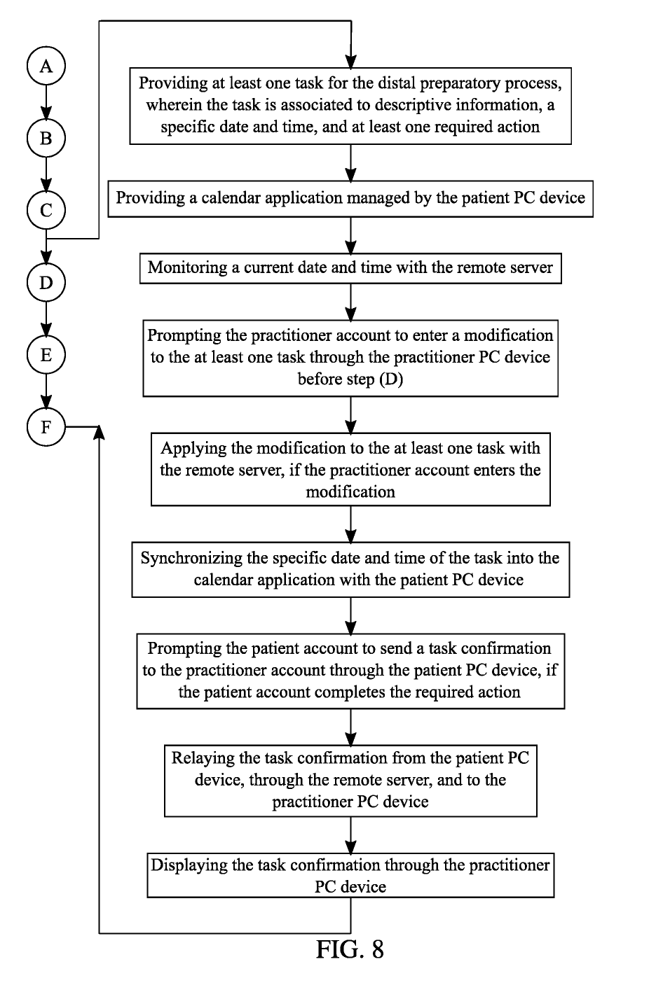

The patient account 3 is able to effectively prepare for the desired medical procedure as at least one task is provided for the distal preparatory process, wherein the task is associated to descriptive information, a specific date and time, and at least one required action, as seen in FIG. 8. With the amount of stress placed on the user of the patient account 3 from the desired medical procedure alone, any necessary tasks that physically and mentally prepare the user of the patient account 3 may be overwhelming and easily overlooked.

The distal preparatory process simplifies the preparation of desired medical procedure for the user of the patient account 3 as a current date and time is monitored with the remote server, also seen in FIG. 8. The patient account 3 is alerted to the task by displaying the descriptive information with the patient PC device 4, if the current date and time matches the specific date and time. Furthermore, the patient account 3 is prompted to send a task confirmation to the practitioner account 1 through the patient PC device 4, if the patient account 3 completes the required action. The user of the practitioner account 1 reviews the preparation of the patient account 3 through the application and the preparation of the patient account 3 is more extensive upon the task confirmation. In order for the user of the practitioner account 1 to review the preparation of the patient account 3, the task confirmation is relayed from the patient PC device 4, through the remote server, and to the practitioner PC device 2. The task confirmation is then displayed through the practitioner PC device 2.

As each user corresponding to the patient account 3 has a unique physical and mental condition associated with a given medical condition, the practitioner account 1 may adjust a task to accommodate the unique physical and mental condition of the user of the patient account 3. As shown in FIG. 8, the practitioner account 1 is prompted to enter a modification to the at least one task through the practitioner PC device 2 before Step D so that the patient account 3 is not requested to fulfill a task that does not help or apply to the user of the patient account 3. Instead, the user of the patient account 3 completes a task that caters to the specific physical and medical condition of the user as the modification is applied to the at least one task with the remote server, if the practitioner account 1 enters the modification.

In order to efficiently manage the tasks with any other important dates and notification already stored on the patient PC device 4, a calendar application is provided and managed by the patient PC device 4, also shown in FIG. 8. The calendar application allows the patient account 3 to input important dates and notifications into the patient PC device 4. The specific date and time of the task is synchronized into the calendar application with the patient PC device 4 in order to simplify the planning and notification delivery of the patient PC device 4.

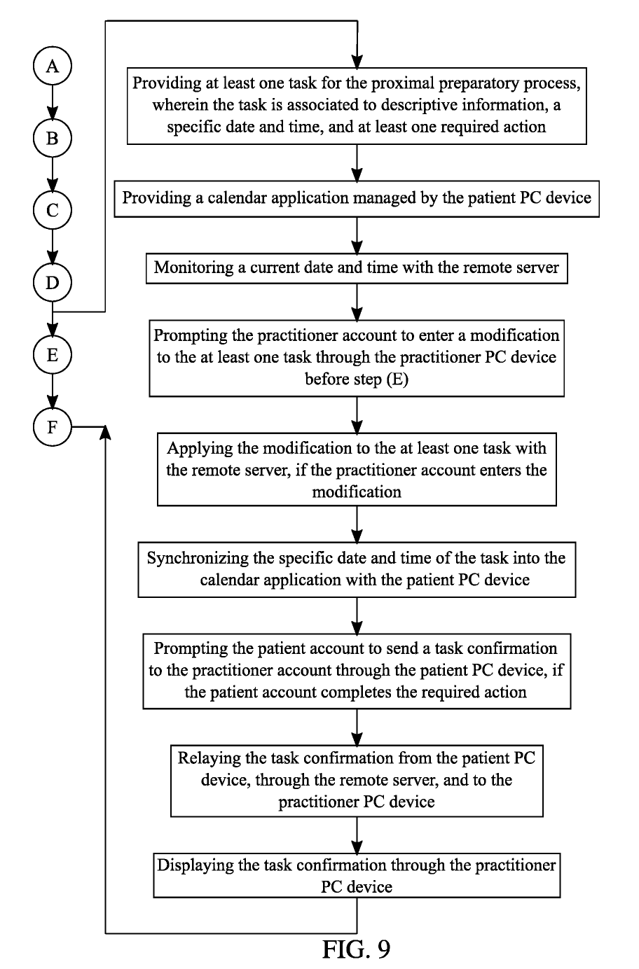

The day of the desired medical procedure imparts substantial stress on the user of the patient account 3. As seen in FIG. 9, the patient account 3 is able to effectively prepare for the desired medical procedure as at least one task is provided for the proximal preparatory process, wherein the task is associated to descriptive information, a specific date and time, and at least one required action. Given the desired medical procedure, the patient account 3 may be requested to complete certain tasks the day of the desired medical procedure. Any task requested the day of the medical procedure is meant to increase the positive results of the desired medical procedure and lessen any risks of the desired medical procedure. Therefore, the tasks requested the day of the desired medical procedure effectively prepare the user physically and mentally without making the user of the patient account 3 feel overwhelming, and consequently easily overlooking the task.

The proximal preparatory process simplifies the day of the desired medical procedure for the patient account 3 as a current date and time is monitored with the remote server. As seen in FIG. 9, the patient account 3 is alerted to the task by displaying the descriptive information with the patient PC device 4, if the current date and time matches the specific date and time. Furthermore, the patient account 3 is prompted to send a task confirmation to the practitioner account 1 through the patient PC device 4, if the patient account 3 completes the required action. The user of the practitioner account 1 reviews the preparation of the patient account 3 through the application and the preparation of the patient account 3 is more extensive upon the task confirmation. In order for the user of the practitioner account 1 to review the preparation of the patient account 3, the task confirmation is relayed from the patient PC device 4, through the remote server, and to the practitioner PC device 2. The task confirmation is then displayed through the practitioner PC device 2.

As each user corresponding to the patient account 3 has a unique physical and mental condition associated with a given medical condition, the practitioner account 1 may adjust a task to accommodate the unique physical and mental condition of the user of the patient account 3, shown in FIG. 9. The practitioner account 1 is prompted to enter a modification to the at least one task through the practitioner PC device 2 before Step E so that the patient account 3 is not requested to fulfill a task that does not help or apply to the user of the patient account 3. Instead, the user of the patient account 3 completes a task that caters to the specific physical and medical condition of the user as the modification is applied to the at least one task with the remote server, if the practitioner account 1 enters the modification.

Similar to that of the distal preparatory process, in order to efficiently manage the tasks with any other important dates and notifications already stored on the patient PC device 4, a calendar application is provided and managed by the patient PC device 4, also shown in FIG. 9. The calendar application allows the patient account 3 to input important dates and notifications into the patient PC device 4. The specific date and time of the task is synchronized into the calendar application with the patient PC device 4 in order to simplify the planning and notification delivery of the patient PC device 4.

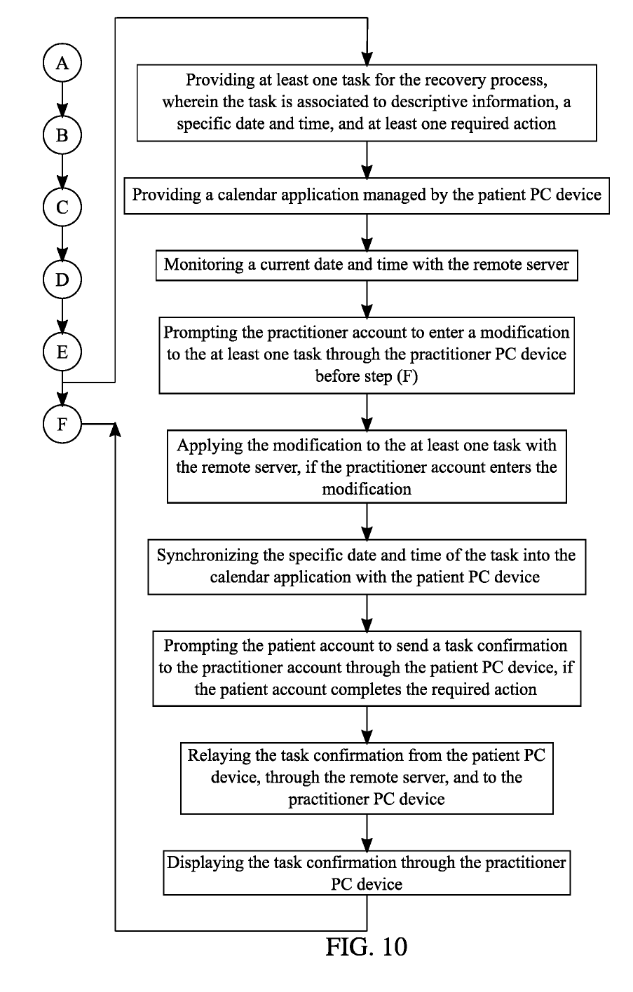

Upon the completion of the desired medical procedure on behalf of the practitioner account 1, the user of the patient account 3 is not only burdened with physical stress but mental stress. The user of the patient account 3 is able to focus solely on healing and resting as at least one task is provided for the recovery process, wherein the task is associated to descriptive information, a specific date and time, and at least one required action, as seen in FIG. 10. Given the desired medical procedure completed, the patient account 3 may be requested to complete certain tasks in order to ensure a quick and effective recovery. Any task requested throughout the recovery process of the medical procedure is meant to facilitate the healing of the user of the patient account 3 and lessen any stress of the user of the patient account 3. The practitioner account 1 is able to better monitor the progress of the user of the patient account 3 through completed tasks by the patient account 3. Therefore, the tasks requested throughout the recovery of the user of the patient account 3 ensures that the user of the patient account 3 reaches a full recovery without the user of the patient account 3 feeling overwhelming. If the user of the patient account 3 feels overwhelmed, the user of the patient account 3 may easily neglect a task and set back the recovery process as a result.

The recovery process facilitates the recovery of the desired medical procedure for the user of the patient account 3 as a current date and time is monitored with the remote server, as seen in FIG. 10. The patient account 3 is alerted to the task by displaying the descriptive information with the patient PC device 4, if the current date and time matches the specific date and time. Furthermore, the patient account 3 is prompted to send a task confirmation to the practitioner account 1 through the patient PC device 4, if the patient account 3 completes the required action. The user of the practitioner account 1 reviews the preparation of the patient account 3 through the application and, consequently, the preparation of the patient account 3 is more extensive upon the task confirmation. In order for the user of the practitioner account 1 to review the preparation of the patient account 3, the task confirmation is relayed from the patient PC device 4, through the remote server, and to the practitioner PC device 2. The task confirmation is then displayed through the practitioner PC device 2, automatically alerting the practitioner account 1 of the completion of the task.

As each user corresponding to the patient account 3 has a unique physical and mental condition associated with a given medical condition, the practitioner account 1 may adjust a task to accommodate the unique physical and mental condition of the user of the patient account 3, shown in FIG. 10. The practitioner account 1 is prompted to enter a modification to the at least one task through the practitioner PC device 2 before Step F so that the patient account 3 is not requested to fulfill a task that does not help or apply to the user of the patient account 3. Instead, the user of the patient account 3 completes a task that caters to the specific physical and medical condition of the user as the modification is applied to the at least one task with the remote server, if the practitioner account 1 enters the modification.

Similar to that of the distal preparatory process and proximal preparatory process, in order to efficiently manage the tasks with any other important dates and notification already stored on the patient PC device 4, a calendar application is provided and managed by the patient PC device 4, also seen in FIG. 10. The calendar application allows the patient account 3 to input important dates and notifications into the patient PC device 4. The specific date and time of the task is synchronized into the calendar application with the patient PC device 4 in order to simplify the planning and notification delivery of the patient PC device 4.

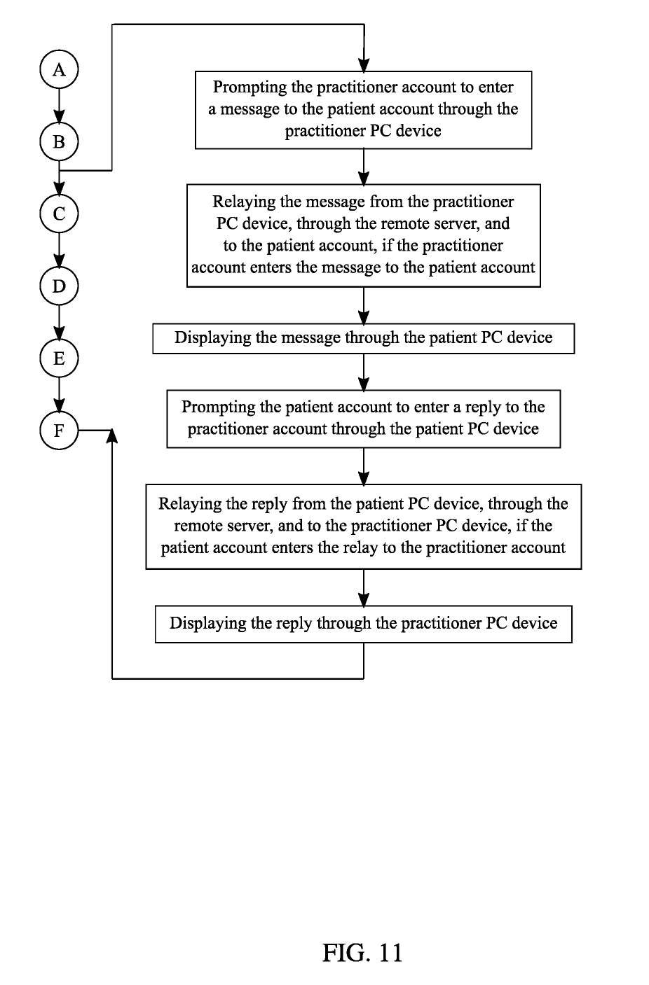

The practitioner account 1 may communicate with the patient account 3 if the user of the practitioner account 1 notices anything unusual with respect to the completed tasks of the patient account 3. In order for the practitioner account 1 to communicate with the patient account 3, the practitioner account 1 is prompted to enter a message to the patient account 3 through the practitioner PC device 2, as shown in FIG. 11. So that the user of the practitioner account 1 and the user of the patient account 3 may communicate with each other without physically being in each other's presence, the message is relayed from the practitioner PC device 2, through the remote server, and to the patient account 3, if the practitioner account 1 enters the message to the patient account 3. The message is viewable by the user of the patient account 3 as the message is displayed through the patient PC device 4. The user of the patient account 3 may respond once the patient account 3 is prompted to enter a reply to the practitioner account 1 through the patient PC device 4. Similarly, the reply from the patient PC device 4 is relayed, through the remoter server, and to the practitioner PC device 2, if the patient account 3 enters the relay to the practitioner account 1. The reply is displayed through the practitioner PC device 2, allowing the user of the patient account 3 to communicate with the user of the practitioner without being in each other's presence.

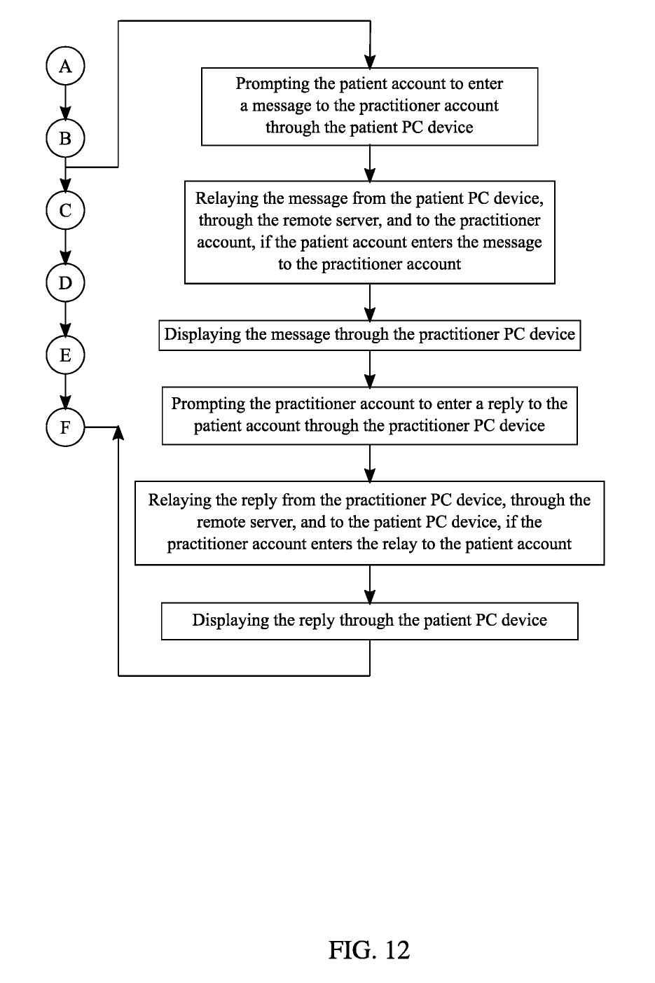

Likewise, the patient account 3 may communicate with the practitioner account 1 if the user of the patient account 3 would like to ask the user of the practitioner account 1 a question or inform the user of the practitioner account 1 about a task or status. In order for the patient account 3 to communicate with the practitioner account 1, the patient account 3 is prompted to enter a message to the practitioner account 1 through the patient PC device 4, seen in FIG. 12. The message is relayed from the patient PC device 4, through the remote server, and to the practitioner account 1, if the patient account 3 enters the message to the practitioner account 1 so that the user of the patient account 3 and the user of the practitioner account 1 may communicate with each other without physically being in each other's presence. The message is viewable by the user of the practitioner account 1 as the message is displayed through the practitioner PC device 2. The user of the practitioner account 1 may respond once the practitioner account 1 is prompted to enter a reply to the patient account 3 through the practitioner PC device 2. Similarly, the reply from the practitioner PC device 2 is relayed, through the remoter server, and to the patient PC device 4, if the practitioner account 1 enters the relay to the patient account 3. The reply is displayed through the patient PC device 4, allowing the user of the practitioner account 1 to communicate with the user of the patient without being in each other's presence.

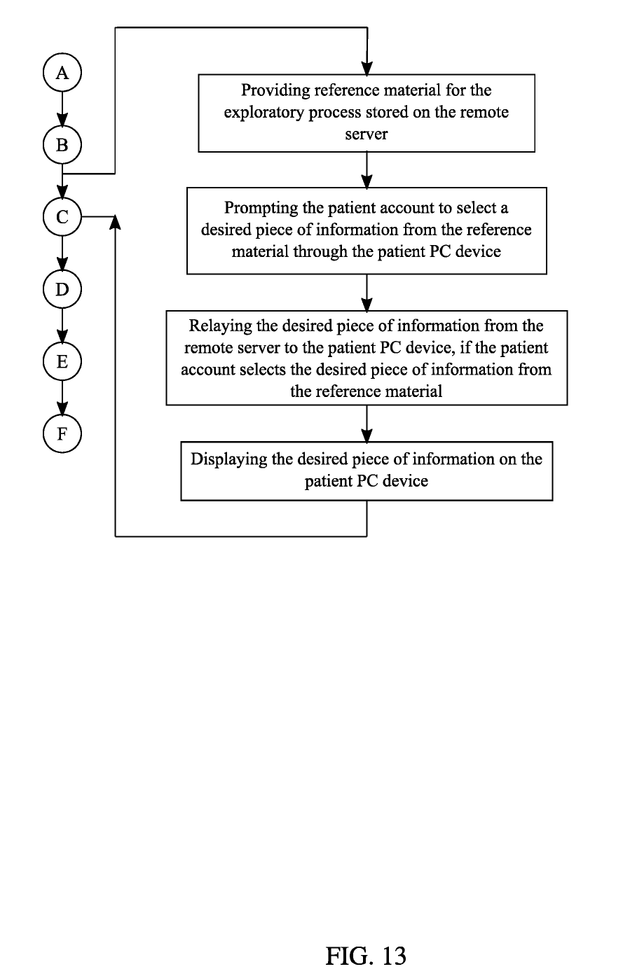

The present invention further simplifies the process of a desired medical procedure by providing the patient account 3 with reference material. The reference material is contoured to the unique user of the patient account 3. The reference material reduces any stress related to searching and filtering of all the data and information regarding related material for the patient account 3. The reference material is also organized with respect to each step. As shown in FIG. 13, reference material for the exploratory process is provided and stored on the remote server. The reference material for the exploratory process allows the user of the patient account 3 to make an informed decision regarding the type of desired medical procedure and if any additional procedures are necessary. In order to view specific reference material, the patient account 3 is prompted to select a desired piece of information from the reference material through the patient PC device 4. The desired piece of information is relayed from the remote server to the patient PC device 4, if the patient account 3 selects the desired piece of information from the reference material. The user of the patient account 3 may view the desired piece of information as the desired piece of information is then displayed on the patient PC device 4.

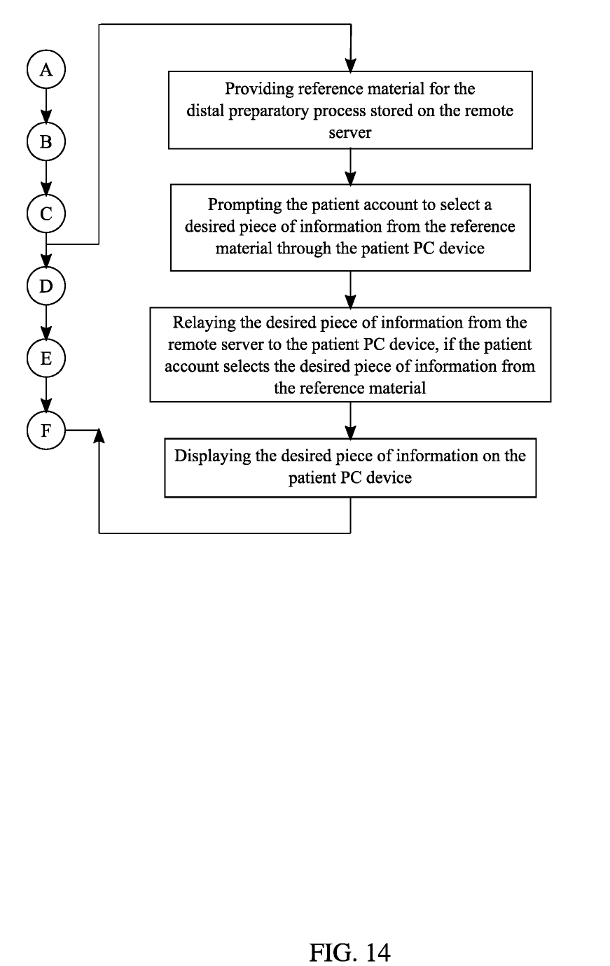

As seen in FIG. 14, the reference material for the distal proximal process allows the user of the patient account 3 to learn more about the status of a patient account 3 before the desired medical procedure and answer any questions regarding the preparation before the desired medical procedure. In order to view specific reference material, the patient account 3 is prompted to select a desired piece of information from the reference material through the patient PC device 4. The desired piece of information is relayed from the remote server to the patient PC device 4, if the patient account 3 selects the desired piece of information from the reference material. The user of the patient account 3 may view the desired piece of information as the desired piece of information is then displayed on the patient PC device 4.

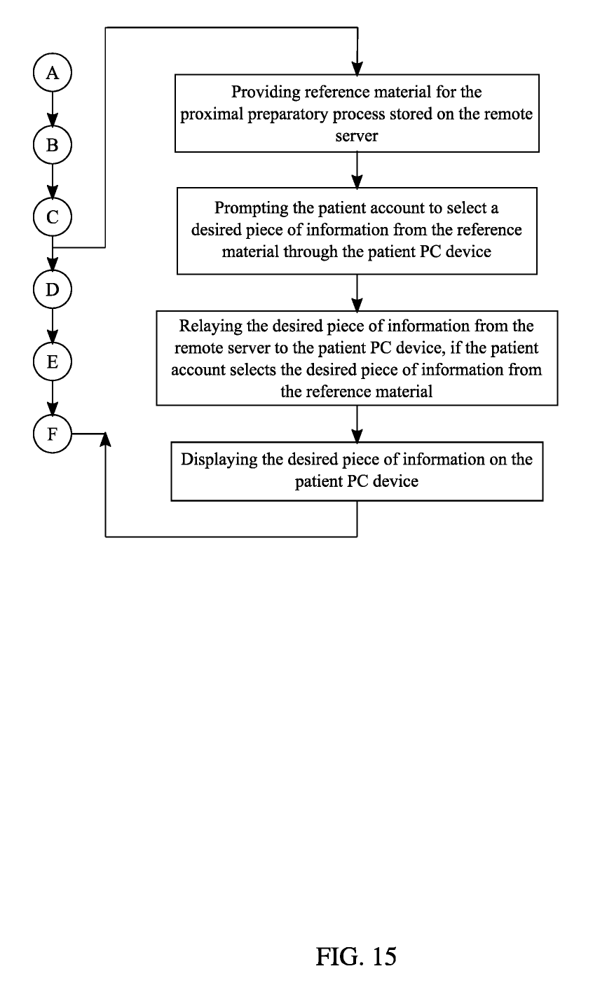

The reference material for the proximal process allows the user of the patient account 3 to learn more and answer common questions regarding the desired medical procedure. Reference material for the proximal process is provided and stored on the remote server, as seen in FIG. 15. In order to view specific reference material, the patient account 3 is prompted to select a desired piece of information from the reference material through the patient PC device 4. The desired piece of information is relayed from the remote server to the patient PC device 4, if the patient account 3 selects the desired piece of information from the reference material. The user of the patient account 3 may view the desired piece of information as the desired piece of information is then displayed on the patient PC device 4.

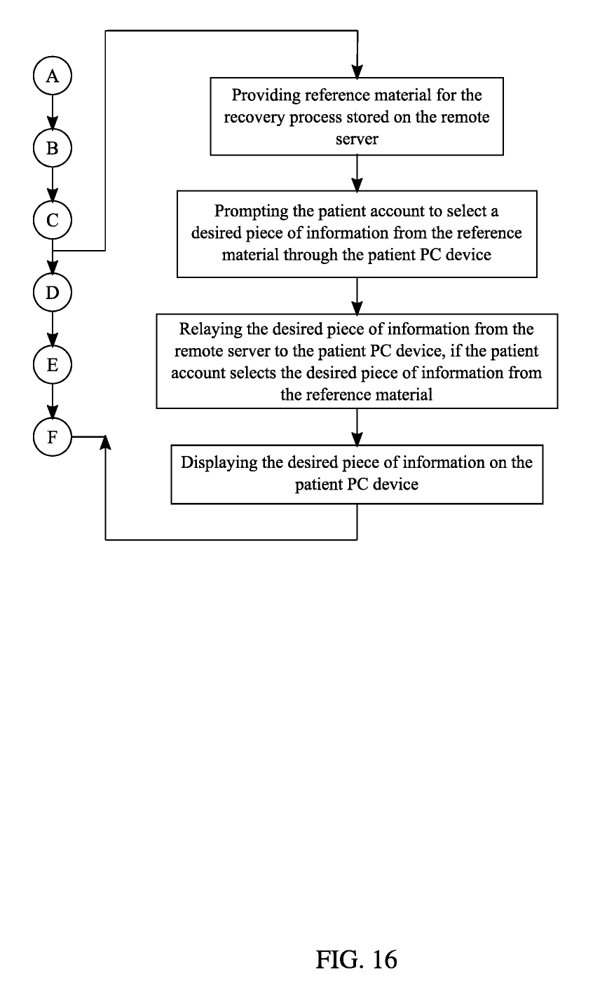

As seen in FIG. 16, the reference material for the recovery process allows the user of the patient account 3 to learn more and answer common questions regarding his or her own recovery with respect to the desired medical procedure. Reference material for the recovery process is provided and stored on the remote server. In order to view specific reference material, the patient account 3 is prompted to select a desired piece of information from the reference material through the patient PC device 4. The desired piece of information is relayed from the remote server to the patient PC device 4, if the patient account 3 selects the desired piece of information from the reference material. The user of the patient account 3 may view the desired piece of information as the desired piece of information is then displayed on the patient PC device 4.

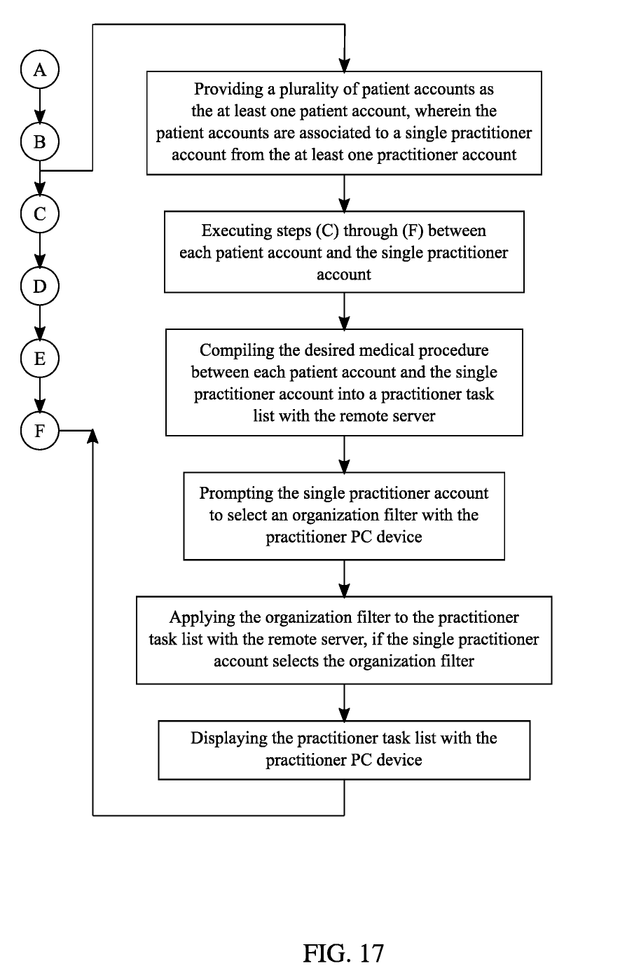

As the user of the practitioner account 1 treats the users of a plurality of patient accounts 3, the practitioner account 1 organizes each patient account 3 for easy viewing, accurate task adjustments, and effective scheduling. More specifically, a plurality of patient accounts 3 is provided as the at least one patient account 3, wherein the patient accounts 3 are associated to a single practitioner account 1 from the at least one practitioner account 1. As seen in FIG. 17, the single practitioner account 1 accommodates to the medical conditions and physical status of the user of the patient account 3 as Steps C through F are executed between each patient account 3 and the single practitioner account 1. The desired medical procedure is compiled between each patient account 3 and the single practitioner account 1 into a practitioner task list with the remote server. The practitioner task list may include, but is not limited to, the date, time, and location of the desired medical procedure of each patient account 3, any scheduled checkups with the user of the patient account 3, and so on. The practitioner task list allows the single practitioner account 1 to effectively monitor each patient account 3. The single practitioner account 1 may sort through the practitioner task list as the single practitioner account 1 is prompted to select an organization filter with the practitioner PC device 2. The organization filter may include, but is not limited to, chronological order, type of task, location of task, and so on. If the single practitioner account 1 selects the organization filter, the organization filter is applied to the practitioner task list with the remote server. The practitioner task list is then displayed according to the organization filter with the practitioner PC device 2.

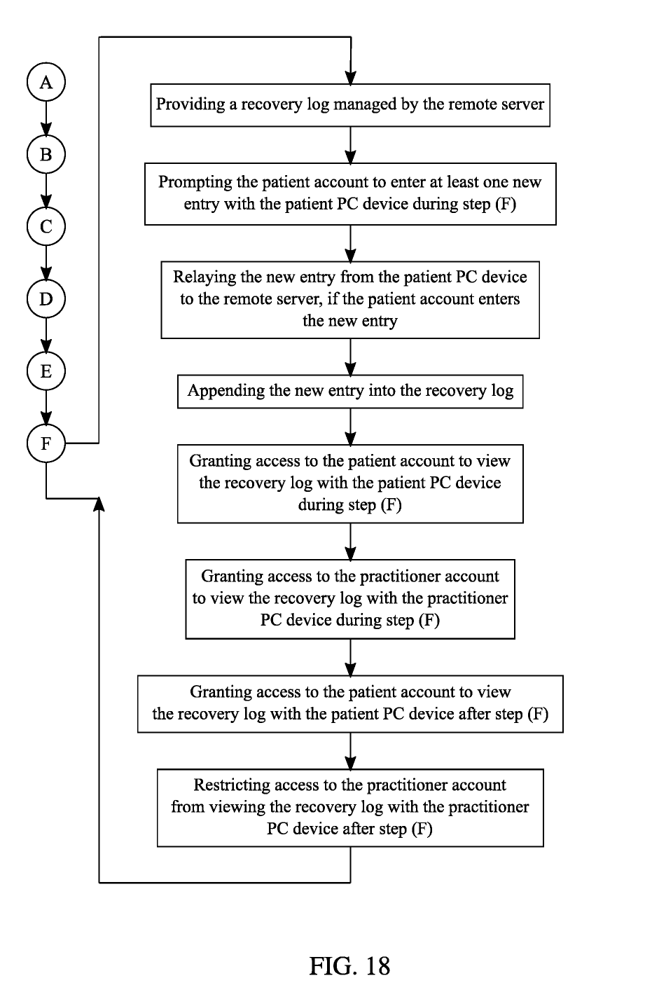

In addition to ensuring the improvement of the physical condition of the user of the patient account 3 via the at least one task for the recovery process, the patient account 3 encourages mental health and strength by holding the user of the patient account 3 accountable for his or her progress. In order to hold the user of the patient account 3 accountable, a recovery log is provided and managed by the remote server, shown in FIG. 18. The patient account 3 is prompted to enter at least one new entry with the patient PC device 4 during Step F. The at least one entry may include details regarding physical improvements, physical difficulties, psychological distress, and so on. If the patient account 3 enters the new entry, the new entry is relayed from the patient PC device 4 to the remote server thereby accounting for the progress of the patient account 3 throughout Step F. The new entry is appended into the recovery log for later review by the patient account 3. The patient account 3 is granted access to view the recovery log with the patient PC device 4 during Step F. Likewise, the practitioner account 1 is granted access to view the recovery log with the practitioner PC device 2 during Step F. The patient account 3 is therefore held accountable and the recovery process is further advanced, and the practitioner account 1 may also monitor the entirety of the recovery process.

Once the recovery process is complete, the patient account 3 may still view the recovery log as the patient account 3 is granted access to view the recovery log with the patient PC device 4 after Step F, also seen in FIG. 18. The patient account 3 may review the recovery log in order to prevent mistakes based on previous desired medical procedures or improve upon completed tasks and mental conditioning throughout each process. The practitioner account 1, however, is restricted access from viewing the recovery log with the practitioner PC device 2 after Step F as the recovery log does not serve as a form of medical records for the patient account 3. Any medical history regarding a patient account 3 is obtained through the traditional means of transferring and sending medical records.

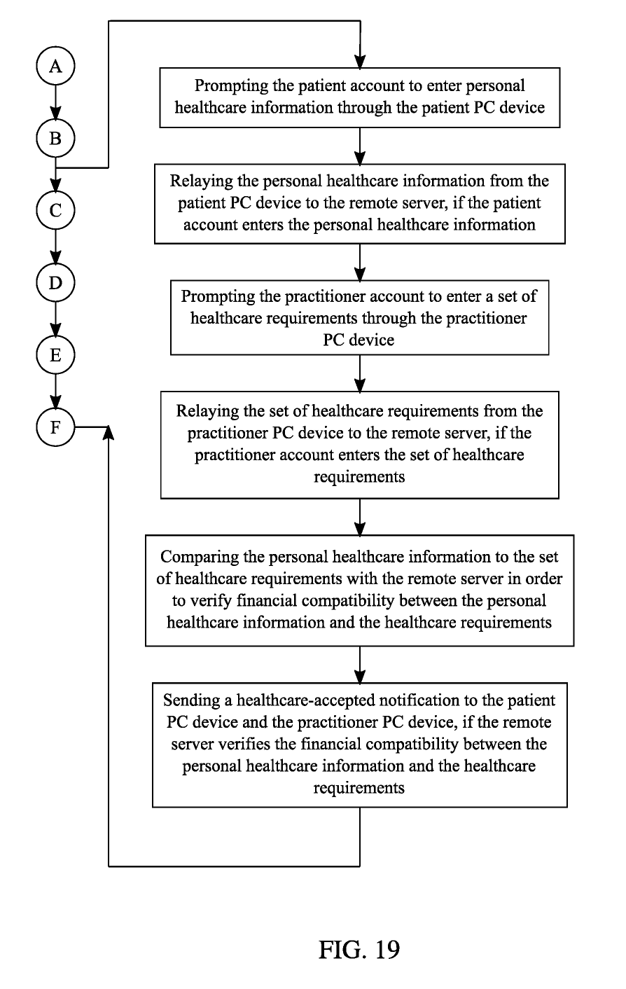

Another major stress imposed on the user of the patient account 3 in addition to the physical stress of the desired medical procedure itself is the financial strain of the desired medical procedure. In order to simplify the financial strain of the desired medical procedure, the patient account 3 is prompted to enter personal healthcare information through the patient PC device 4, seen in FIG. 19. The personal healthcare information includes all health insurance accounts associated with the patient account 3 as the health insurance accounts of the patient account 3 affects a bill or a balance associated with the patient account 3. If the patient account 3 enters the personal healthcare information, the personal healthcare information is relayed from the patient PC device 4 to the remote server. The personal healthcare information is stored on the remote server and is automatically associated with the patient account 3 so that the personal healthcare information is applied to future desired medical procedures.

Similar to standard office procedures of medical offices, the personal healthcare information, such as health insurance, may not be accepted by all practitioner accounts 1. In order to determine if the personal healthcare information is accepted by the practitioner account 1, the practitioner account 1 is first prompted to enter a set of healthcare requirements through the practitioner PC device 2, also seen in FIG. 19. If the practitioner account 1 enters the set of healthcare requirements, the set of healthcare requirements is relayed from the practitioner PC device 2 to the remote server. The set of healthcare requirements is stored for comparison to multiple personal healthcare information of current and further patient accounts 3 associated with the practitioner account 1. The personal healthcare information is compared to the set of healthcare requirements with the remote server in order to verify financial compatibility between the personal healthcare information and the healthcare requirements. If the remote server verifies the financial compatibility between the personal healthcare information and the healthcare requirements, a healthcare-accepted notification is sent to the patient PC device 4 and the practitioner PC device 2. Both the patient account 3 and the practitioner account 1 do not need to process the set of healthcare requirements and the personal healthcare information, respectively, as the remote server compares and verifies the information of patient account 3. The healthcare-accepted notification preferably includes which medical procedures are covered by the health insurance accounts of the patient account 3 and the remaining balance that is charged to the patient account 3.

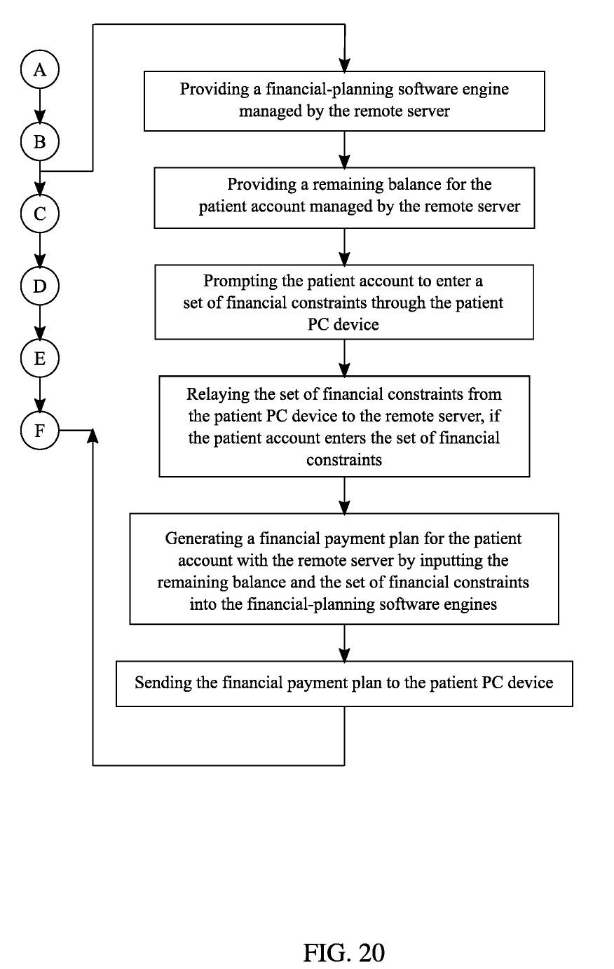

In addition to the comparison and verification of the personal healthcare information of the patient account 3, a financial-planning software engine is provided and managed by the remote server, seen in FIG. 20. A remaining balance for the patient account 3 is also provided and managed by the remote server. The financial-planning software presents the patient account 3 with multiple methods and possibilities of paying the remaining balance of a desired medical procedure. The remaining balance alerts the patient account 3 of the financial amount due to the practitioner account 1. In order to effectively and thoroughly analyze and present the patient account 3 with multiple methods and possibilities of paying the remaining balance, the patient account 3 is first prompted to a set of financial constraints through the patient PC device 4. The set of financial constraints may include monthly income, existing debts and bills, and so on. If the patient account 3 enters the set of financial constraints, the set of financial constraints is relayed from the patient PC device 4 to the remote server. Consequently, a financial plan is generated for the patient account 3 with the remote server by inputting the remaining balance and the set of financial constraints into the financial-planning software engine. The financial payment plan is then sent to the patient PC device 4 so that the user of the patient account 3 may view and select the best option given the set of financial constraints.

Although the invention has been explained in relation to its preferred embodiment, it is to be understood that many other possible modifications and variations can be made without departing from the spirit and scope of the invention as hereinafter claimed.

What is claimed is:

1. A system and method of managing and coordinating a medical procedure, the method comprises the steps of:
A. providing at least one practitioner account managed by at least one remote server, wherein the practitioner account is associated to a practitioner personal computing (PC) device;
B. providing at least one patient account managed by the remote server, wherein the patient account is associated to a patient personal computing (PC) device;
C. executing an exploratory process between the practitioner account and the patient account with the remote server in order to identify a desired medical procedure for the patient account;
D. executing a distal preparatory process between the practitioner account and the patient account in order to gather and manage pre-procedure logistic data for the desired medical procedure;
E. executing a proximal preparatory process between the practitioner account and the patient account in order to gather and manage pre-procedure checklist data for the desired medical procedure; and
F. executing a recovery process between the practitioner account and the patient account in order to gather and manage post-procedure logistic data for the desired medical procedure.

2. The system and method of managing and coordinating a medical procedure, the method as claimed in claim 1 comprises the steps of:
providing at least one administrator account managed by the remote server, wherein the administrator account is associated to an administrator PC device;
providing the practitioner account with an unverified status on the remote server;
prompting the practitioner account to enter a set of credentials through the practitioner PC device;
relaying the set of credentials from the practitioner PC device, through the remote server, and to the administrator PC device;
prompting the administrator account to authenticate or deny the set of credentials with the administrator PC device; and,
updating the practitioner account from the unverified status to a verified status on the remote server, if the administrator account authenticates the set of credentials.

3. The system and method of managing and coordinating a medical procedure, the method as claimed in claim 2 comprises the steps of:
prompting the practitioner account to complete a practitioner profile with the practitioner PC device, if the practitioner PC device is updated from the unverified status to the verified status;
relaying the practitioner profile from the practitioner PC device to the remote server, if the practitioner account completes the practitioner profile; and,
granting access to the patient account to view the practitioner profile with the patient PC device.

4. The system and method of managing and coordinating a medical procedure, the method as claimed in claim 1 comprises the steps of:
prompting the practitioner account to send an engagement invitation to the patient account through the practitioner PC device;
relaying the engagement invitation from the practitioner PC device, through the remote server, and to the patient PC device;
prompting the patient account to accept or deny the engagement invitation through the patient PC device; and,
initiating step (C) with the remote server, if the patient account accepts the engagement invitation.

5. The system and method of managing and coordinating a medical procedure, the method as claimed in claim 1 comprises the steps of:
prompting the practitioner account to confirm or deny a completed status for the desired medical procedure through the practitioner PC device after step (F); and,
designating the desired medical procedure as the completed status with the remote server, if the practitioner account confirms the completed status.

6. The system and method of managing and coordinating a medical procedure, the method as claimed in claim 5 comprises the steps of:
compiling collected data during steps (C) through (F) into a case study for the desired medical procedure with the remote server, if the practitioner account confirms the completed status;
archiving the case study on the remote server;
granting access to the patient account to view the case study with the patient PC device after step (F); and,
restricting access to the practitioner account from viewing the case study with the practitioner PC device after step (F).

7. The system and method of managing and coordinating a medical procedure, the method as claimed in claim 1 comprises the steps of:
providing the at least one practitioner account as a plurality of practitioner accounts, wherein each practitioner account is associated to an agency profile;
prompting the practitioner account to send an agency recommendation to the patient account through the practitioner PC device during steps (C), (D), and (F), wherein the agency recommendation is associated to another account from the plurality of practitioner accounts;
relaying the agency recommendation from the practitioner PC device, through the remote server, and to the patient PC device;
displaying the agency profile of the other account through the patient PC device;
prompting the patient account to accept or deny the agency recommendation through the patient PC device; and, repeating steps (C) through (F) with the other account as the practitioner account, if the patient account accepts the agency recommendation.

8. The system and method of managing and coordinating a medical procedure, the method as claimed in claim 1 comprises the steps of:
providing at least one task for the distal preparatory process, wherein the task is associated to descriptive information, a specific date and time, and at least one required action;
monitoring a current date and time with the remote server;
alerting the patient account to the task by displaying the descriptive information with the patient PC device, if the current date and time matches the specific date and time;
prompting the patient account to send a task confirmation to the practitioner account through the patient PC device, if the patient account completes the required action;
relaying the task confirmation from the patient PC device, through the remote server, and to the practitioner PC device; and,
displaying the task confirmation through the practitioner PC device.

9. The system and method of managing and coordinating a medical procedure, the method as claimed in claim 8 comprises the steps of:
prompting the practitioner account to enter a modification to the at least one task through the practitioner PC device before step (D); and,
applying the modification to the at least one task with the remote server, if the practitioner account enters the modification.

10. The system and method of managing and coordinating a medical procedure, the method as claimed in claim 8 comprises the steps of:
providing a calendar application managed by the patient PC device; and,
synchronizing the specific date and time of the task into the calendar application with the patient PC device.

11. The system and method of managing and coordinating a medical procedure, the method as claimed in claim 1 comprises the steps of:
providing at least one task for the proximal preparatory process, wherein the task is associated to descriptive information, a specific date and time, and at least one required action;
monitoring a current date and time with the remote server;
alerting the patient account to the task by displaying the descriptive information with the patient PC device, if the current date and time matches the specific date and time;
prompting the patient account to send a task confirmation to the practitioner account through the patient PC device, if the patient account completes the required action;
relaying the task confirmation from the patient PC device, through the remote server, and to the practitioner PC device; and,
displaying the task confirmation through the practitioner PC device.

12. The system and method of managing and coordinating a medical procedure, the method as claimed in claim 11 comprises the steps of:
prompting the practitioner account to enter a modification to the at least one task through the practitioner PC device before step (E); and,
applying the modification to the at least one task with the remote server, if the practitioner account enters the modification.

13. The system and method of managing and coordinating a medical procedure, the method as claimed in claim 11 comprises the steps of:
providing a calendar application managed by the patient PC device; and,
synchronizing the specific date and time of the task into the calendar application with the patient PC device.

14. The system and method of managing and coordinating a medical procedure, the method as claimed in claim 1 comprises the steps of:
providing at least one task for the recovery process, wherein the task is associated to descriptive information, a specific date and time, and at least one required action;
monitoring a current date and time with the remote server;
alerting the patient account to the task by displaying the descriptive information with the patient PC device, if the current date and time matches the specific date and time;
prompting the patient account to send a task confirmation to the practitioner account through the patient PC device, if the patient account completes the required action;
relaying the task confirmation from the patient PC device, through the remote server, and to the practitioner PC device; and,
displaying the task confirmation through the practitioner PC device.

15. The system and method of managing and coordinating a medical procedure, the method as claimed in claim 14 comprises the steps of:
prompting the practitioner account to enter a modification to the at least one task through the practitioner PC device before step (F); and,
applying the modification to the at least one task with the remote server, if the practitioner account enters the modification.

16. The system and method of managing and coordinating a medical procedure, the method as claimed in claim 14 comprises the steps of:
providing a calendar application managed by the patient PC device; and,
synchronizing the specific date and time of the task into the calendar application with the patient PC device.

17. The system and method of managing and coordinating a medical procedure, the method as claimed in claim 1 comprises the steps of:
prompting the practitioner account to enter a message to the patient account through the practitioner PC device;
relaying the message from the practitioner PC device, through the remote server, and to the patient account, if the practitioner account enters the message to the patient account;
displaying the message through the patient PC device;
prompting the patient account to enter a reply to the practitioner account through the patient PC device;
relaying the reply from the patient PC device, through the remote server, and to the practitioner PC device, if the patient account enters the relay to the practitioner account; and,
displaying the reply through the practitioner PC device.

18. The system and method of managing and coordinating a medical procedure, the method as claimed in claim 1 comprises the steps of:
prompting the patient account to enter a message to the practitioner account through the patient PC device;
relaying the message from the patient PC device, through the remote server, and to the practitioner account, if the patient account enters the message to the practitioner account;
displaying the message through the practitioner PC device;
prompting the practitioner account to enter a reply to the patient account through the practitioner PC device;
relaying the reply from the practitioner PC device, through the remote server, and to the patient PC device, if the practitioner account enters the relay to the patient account; and,
displaying the reply through the patient PC device.

19. The system and method of managing and coordinating a medical procedure, the method as claimed in claim 1 comprises the steps of:
providing reference material for the exploratory process stored on the remote server;
prompting the patient account to select a desired piece of information from the reference material through the patient PC device;
relaying the desired piece of information from the remote server to the patient PC device, if the patient account selects the desired piece of information from the reference material; and,
displaying the desired piece of information on the patient PC device.

20. The system and method of managing and coordinating a medical procedure, the method as claimed in claim 1 comprises the steps of:
providing reference material for the distal preparatory process stored on the remote server;
prompting the patient account to select a desired piece of information from the reference material through the patient PC device;
relaying the desired piece of information from the remote server to the patient PC device, if the patient account selects the desired piece of information from the reference material; and,
displaying the desired piece of information on the patient PC device.

21. The system and method of managing and coordinating a medical procedure, the method as claimed in claim 1 comprises the steps of:
providing reference material for the proximal preparatory process stored on the remote server;
prompting the patient account to select a desired piece of information from the reference material through the patient PC device;
relaying the desired piece of information from the remote server to the patient PC device, if the patient account selects the desired piece of information from the reference material; and,
displaying the desired piece of information on the patient PC device.

22. The system and method of managing and coordinating a medical procedure, the method as claimed in claim 1 comprises the steps of:
providing reference material for the recovery process stored on the remote server;
prompting the patient account to select a desired piece of information from the reference material through the patient PC device;
relaying the desired piece of information from the remote server to the patient PC device, if the patient account selects the desired piece of information from the reference material; and,
displaying the desired piece of information on the patient PC device.

23. The system and method of managing and coordinating a medical procedure, the method as claimed in claim 1 comprises the steps of:
providing a plurality of patient accounts as the at least one patient account, wherein the patient accounts are associated to a single practitioner account from the at least one practitioner account;
executing steps (C) through (F) between each patient account and the single practitioner account;
compiling the desired medical procedure between each patient account and the single practitioner account into a practitioner task list with the remote server;
prompting the single practitioner account to select an organization filter with the practitioner PC device;
applying the organization filter to the practitioner task list with the remote server, if the single practitioner account selects the organization filter; and,
displaying the practitioner task list with the practitioner PC device.

24. The system and method of managing and coordinating a medical procedure, the method as claimed in claim 1 comprises the steps of:
providing a recovery log managed by the remote server;
prompting the patient account to enter at least one new entry with the patient PC device during step (F);
relaying the new entry from the patient PC device to the remote server, if the patient account enters the new entry;
appending the new entry into the recovery log;
granting access to the patient account to view the recovery log with the patient PC device during step (F); and,
granting access to the practitioner account to view the recovery log with the practitioner PC device during step (F).

25. The system and method of managing and coordinating a medical procedure, the method as claimed in claim 24 comprises the steps of:
granting access to the patient account to view the recovery log with the patient PC device after step (F); and,
restricting access to the practitioner account from viewing the recovery log with the practitioner PC device after step (F).

26. The system and method of managing and coordinating a medical procedure, the method as claimed in claim 1 comprises the steps of:
prompting the patient account to enter personal healthcare information through the patient PC device;
relaying the personal healthcare information from the patient PC device to the remote server, if the patient account enters the personal healthcare information;
prompting the practitioner account to enter a set of healthcare requirements through the practitioner PC device;
relaying the set of healthcare requirements from the practitioner PC device to the remote server, if the practitioner account enters the set of healthcare requirements;
comparing the personal healthcare information to the set of healthcare requirements with the remote server in order to verify financial compatibility between the personal healthcare information and the healthcare requirements; and,
sending a healthcare-accepted notification to the patient PC device and the practitioner PC device, if the remote server verifies the financial compatibility between the personal healthcare information and the healthcare requirements.

27. The system and method of managing and coordinating a medical procedure, the method as claimed in claim 1 comprises the steps of:
providing a financial-planning software engine managed by the remote server;
providing a remaining balance for the patient account managed by the remote server;
prompting the patient account to enter a set of financial constraints through the patient PC device;
relaying the set of financial constraints from the patient PC device to the remote server, if the patient account enters the set of financial constraints;
generating a financial payment plan for the patient account with remote server by inputting the remaining balance and the set of financial constraints into the financial-planning software engine; and,
sending the financial payment plan to the patient PC device.
